(19) United States
(12) Patent Application Publication
(10) Pub. No.: US 2014/0200622 A1
(43) Pub. Date: Jul. 17, 2014

(54) Combination Device and Methods for Treating Congestive Heart Failure and Stroke

(71) Applicants: Reese S. Terry, JR., Houston, TX (US); Ingela Danielsson, Seabrook, TX (US)

(72) Inventors: Reese S. Terry, JR., Houston, TX (US); Ingela Danielsson, Seabrook, TX (US)

(73) Assignee: Trifectas Medical Corp., Seabrook, TX (US)

(21) Appl. No.: 13/744,155

(22) Filed: Jan. 17, 2013

(51) Int. Cl. A61N 1/36

(52) U.S. Cl. CPC A61N 1/36053
USPC 607/11

(57) ABSTRACT

According to some embodiments, the present invention provides a device, system, and methods for treating a plurality of conditions in a patient, where the device, system, and methods provide a stimulation signal for each condition, wherein each stimulation signal has one or more of polarity, direction, and pulse patterns selected for the respective condition. According to some embodiments, the conditions comprise congestive heart failure and stroke.

### CROSS REFERENCE TO RELATED APPLICATIONS

This application claims priority to U.S. Provisional Application Ser. No. 61/587,553, filed Jan. 17, 2012, hereby incorporated herein by reference.

### BACKGROUND OF THE INVENTION

There has been interest in devices and methods for treating medical conditions with vagus nerve stimulation. An individual has two vagus nerves, each one a cranial nerve, and each extending from the brain through the chest to the abdomen. Each vagus nerve has a multiple branches. Typical devices are implantable.

U.S. Pat. No. 4,573,481 to Bullara describes an electrode array. More particularly, U.S. Pat. No. 4,573,481 describes an implantable helical electrode assembly configured to fit around a nerve for electrically triggering or measuring an action potential or for blocking conduction in nerve tissue. U.S. Pat. No. 4,573,481 discloses that external controls can be used to command implanted nerve stimulators to regain muscle control in injured limbs, to control bladder and sphincter function, to alleviate pain and hypertension, and to restore proper function to many other portions of an impaired or injured nerve-muscle system.

U.S. Pat. No. 5,304,206 to Baker, Jr., et al. describes activation techniques for an implantable medical device. More particularly, U.S. Pat. No. 5,304,206 describes neurostimulators implemented to respond to a patient initiated signal which may be derived either manually or automatically to selectively activate the stimulus generator. U.S. Pat. No. 5,304,206 discloses that the neurostimulators may be manually activated with a magnet or with a programming wand for telemetry of changes to software parameters, and use of neurostimulators for treating or controlling medical, psychiatric or neurological disorders by application of modulating electrical signals to a selected nerve or nerves of the patient.

There has been interest in implantable medical devices to treat congestive heart failure. Congestive heart failure is a common and significant medical condition. For example, Schocken, et al., J. Am. Coll. Cardiol. 1992; 20(2): 301-6. report that congestive heart failure has a prevalence of 2% of the United States population and is associated with increased mortality risk.

U.S. Pat. No. 6,622,041 to Terry, Jr., et al. describes treatment of congestive heart failure and autonomic cardiovascular drive disorders. More particularly, U.S. Pat. No. 6,622,041 describes a device for treating patients suffering from congestive heart failure that includes an implantable neurostimulator for stimulating the patient's vagus nerve at or above the cardiac branch with an electrical pulse waveform at a stimulating rate sufficient to maintain the patient's heart beat at a rate well below the patient's normal resting heart rate, thereby allowing rest and recovery of the heart muscle, to increase in coronary blood flow, and/or growth of coronary capillaries.

There has also been interest in medical devices to treat stroke. US 20100069995 to Danielsson describes a method for improving functional recovery after stroke by electrical stimulation of a cranial nerve. More particularly, US 20100069995 describes applying to a first cranial nerve a first stimulating electrical signal optimized so as to promote stroke recovery, and optionally applying to one or more of the first cranial nerve and a second cranial nerve a second stimulating electrical signal optimized so as to control a concomitant dysfunction. US 20100069995 discloses that concomitant dysfunctions include, but are not limited to, disabilities introduced by stroke and co-morbid disorders.

Studies have found that persons with congestive heart failure are at increased risk of stroke. For example, a study of 630 by the Mayo clinic presented in 2005 at the American Heart Association meeting found that the risk of stroke increased by 17-fold following a diagnosis of CHF and stayed elevated during the five year follow-up of the study. (Am Heart J. 2006 July; 152(1):102-9).

Stimulation parameters and polarity typically differ between treatment of congestive heart failure and treatment of stroke. The directionality of vagus nerve stimulation is opposite for congestive heart failure and stroke. In congestive heart failure, the goal is to direct the stimulation toward the heart and minimize the stimulation of the brain, whereas the goal in stroke is to direct the signal toward the brain and minimize the signal towards the heart. Moreover, the signal amplitude is typically much higher (4 mA typical) for treat congestive heart failure than for stroke (1 mA typical) and the stimulation frequency, ON time and OFF may be different.

There has been interest in treating single disorders with unidirectional stimulation. U.S. Pat. No. 6,684,105 to Cohen et al. describes treatment of disorders by unidirectional stimulation. More particularly, U.S. Pat. No. 6,684,105 describes an electrode device adapted to be coupled to longitudinal nervous tissue of the subject, and a control unit is adapted to drive the electrode device to apply to the nervous tissue a current which is capable of inducing action potentials that propagate in the nervous tissue in a first direction, so as to treat the condition. U.S. Pat. No. 6,684,105 disclose that the control unit is further adapted to suppress action potentials from propagating in the nervous tissue in a second direction opposite to the first direction. U.S. Pat. No. 6,684,105 describes treating one or more of the following exemplary conditions of the subject: a sleep disorder, a gastrointestinal motility disorder, an eating disorder, obesity, anorexia, a gastrointestinal tract disorder, hypertension, coma, or epilepsy by inducing afferent nerve impulses. U.S. Pat. No. 6,684,105 discloses suppressing (a) increased acid secretion in a gastrointestinal tract of the subject, (b) muscular contraction, and/or (c) bradycardia by suppressing efferent nerve impulses.

U.S. Pat. No. 7,908,008 to Ben-David, et al., describes treatment for disorders by parasympathetic stimulation. More particularly, U.S. Pat. No. 7,908,008 describes a treatment method, including identifying a subject as one who is selected to undergo an interventional medical procedure, and, in response to the identifying, reducing a likelihood of a potential adverse effect of the procedure by applying an electrical current to a parasympathetic site of the subject selected from the group consisting of: a vagus nerve of the subject, an epicardial fat pad of the subject, a pulmonary vein of the subject, a carotid artery of the subject, a carotid sinus of the subject, a coronary sinus of the subject, a vena cava vein of the subject, a jugular vein of the subject, a right ventricle of the subject, a parasympathetic ganglion of the subject, and a parasympathetic nerve of the subject U.S. Pat. No. 7,908,008 discloses treating a heart condition by inducing efferent nerve impulses and suppressing afferent nerve impulses toward the brain in order to minimize unintended side effects.

There has been interest in treating single disorders with switching polarity. US 20100023090 to Jaax, et al. describes a system and method for avoiding, reversing, and managing neurological accommodation to electrical stimulation. More particularly, US 20100023090 describes a method for programming a neurostimulation device that comprises outputting a pulsed electrical waveform from the neuro stimulation device between a plurality of electrodes while at least one of the electrodes has a first polarity, thereby stimulating neural tissue adjacent the at least one electrode, allowing the neural tissue to undergo neurological accommodation in response to the electrical energy output between the electrodes, switching the electrode(s) from the first polarity to a second polarity (which may be automatically initiated), and outputting the pulsed electrical waveform from the neurostimulation device between the electrodes while the electrode(s) has the second polarity, thereby hyperpolarizing the neural tissue to reverse the neurological accommodation, and switching the electrode(s) from the second polarity to the first polarity (which may be automatically initiated), and outputting the pulsed electrical waveform from the neurostimulation device between the electrodes while the electrode(s) has the first polarity, thereby stimulating the previously hyperpolarized neural tissue. US 20100023090 discloses that the invention may be used with any type of implantable electrical circuitry used to stimulate tissue.

US 20110213437 to Armstrong, et al. describes changeable electrode polarity stimulation by implantable medical device. More particularly, US 20110213437 describes a method of treating a medical condition in a patient using an implantable medical device including coupling at least a first electrode and a second electrode to a cranial nerve of the patient, providing a programmable electrical signal generator coupled to the first electrode and the second electrode, generating a first electrical signal with the electrical signal generator, applying the first electrical signal to the electrodes, wherein the first electrode is a cathode and the second electrode is an anode, reversing the polarity of the first electrode and the second electrode, yielding a configuration wherein the first electrode is an anode and the second electrode is a cathode, generating a second electrical signal with the electrical signal generator, applying the second electrical signal to the electrodes, reversing the polarity of the first electrode and the second electrode, yielding a configuration wherein the first electrode is a cathode and the second electrode is an anode, generating a third electrical signal with the electrical signal generator, and applying the third electrical signal to the electrodes. US 20110213437 discloses a need for apparatuses and methods stimulating both the brain and an organ outside the brain. US 20110213437 discloses that the first signal may have a direction and polarity in which action potentials to the brain are not blocked and action potentials to a distal region are blocked, and that the second signal may have a direction and polarity in which action potentials to the brain are blocked and action potentials to a distal region are not blocked, and that the third signal may have the direction and polarity of the first signal. US 20110213437 discloses that the medical condition may be selected from among neurological disorders, neuropsychiatric disorders, a cardiac disorders, endocrine disorders, gastrointestinal disorders, motility disorders, pain, traumatic brain injury, coma, and hypertension.

Notwithstanding the above-described teachings, devices and methods that could be used to treat congestive heart failure and stroke in combination would be highly desirable.

### BRIEF SUMMARY OF THE INVENTION

According to some embodiments, the present invention provides a device, system, and methods for treating a plurality of conditions in a patient, where the device, system, and methods provide a stimulation signal for each condition, wherein each stimulation signal has one or more of polarity, direction, and pulse patterns selected for the respective condition. According to some embodiments, the conditions comprise congestive heart failure and stroke.

According to one aspect, a single bipolar electrode implantable medical device for treating a plurality of conditions of a patient, comprises a stimulation electrode assembly attachable to a vagus nerve, wherein the stimulation electrode assembly comprises first and second electrodes; a polarity reversal unit in electrical communication with the stimulation electrode assembly; and a stimulation unit in electrical communication with the polarity reversal unit, wherein the stimulation unit is capable to control interleaved signals of a first stimulation pattern transmittable by the first electrode and a second stimulation pattern transmittable by the second electrode.

According to another aspect, a single nerve dual bipolar electrode implantable medical device for treating a plurality of conditions in a patient comprises a first stimulation electrode assembly attachable to a vagus nerve, wherein the first stimulation electrode assembly has a first polarity; a second stimulation electrode assembly attachable to the vagus nerve, wherein the second stimulation electrode assembly has a second polarity; a first stimulation unit in electrical communication with the first stimulation electrode assembly, wherein the first stimulation unit is capable to control a first directional stimulation pattern transmittable by the first stimulation electrode assembly; and a second stimulation unit in electrical communication with the second directional stimulation electrode assembly, wherein the second stimulation unit is capable to control a second directional stimulation pattern transmittable by the second stimulation electrode assembly.

According to yet another aspect, an left and right nerve dual bipolar electrode implantable medical device comprises a first stimulation electrode assembly attachable to a first vagus nerve, wherein the first stimulation electrode assembly has a first polarity; a second stimulation electrode assembly attachable to a second vagus nerve, wherein the second stimulation electrode assembly as a second polarity; a first stimulation unit in electrical communication with the first stimulation electrode assembly, wherein the first stimulation unit is capable to control a first directional stimulation pattern transmittable by the first stimulation electrode assembly; and a second stimulation unit in electrical communication with the second directional stimulation electrode assembly, wherein the second stimulation unit is capable to control a second directional stimulation pattern transmittable by the second stimulation electrode assembly.

According to any of the above-described aspects, the implantable medical device may further comprise a signal control processor in electrical communication with each stimulation unit.

According to any of the above-described aspects, the first stimulation pattern may comprise a heart stimulation pattern; and wherein the second stimulation pattern comprises a brain stimulation pattern.

According to any of the above-described aspects, the implantable medical device may further comprises an EKG sensing electrode member; and an EKG processor in electrical communication with the EKG sensing electrode member. The implantable medical device may further comprise a signal control processor in electrical communication with each stimulation unit and the EKG processor.

A system for treating a plurality of conditions in a patient may comprise the implantable medical device according to any one of the above-described aspects and a handheld programming wand, wherein the handheld programming wand is capable to change one or both of the first stimulation pattern and the second stimulation pattern.

A treatment method for treating a plurality of conditions in a patient may comprise providing an implantable medical device according to any one of the above-described aspects. The method may further comprise providing a handheld programming wand is capable to change one or both of the first stimulation pattern and the second stimulation pattern. The method may further comprise selecting the first stimulation pattern to stimulate the heart and selecting the second stimulation pattern to stimulate the brain.

A single bipolar electrode treatment method for treating a plurality of conditions in a patient may comprise attaching a stimulation electrode assembly to a vagus nerve in the patient; stimulating the vagus nerve with a first stimulation pattern transmitting by the electrode; changing the polarity of the electrode; and stimulating the vagus nerve with a second stimulation pattern transmitted by the electrode.

A single nerve dual bipolar electrode treatment method for treating a plurality of conditions in a patient may comprise attaching a first stimulation electrode assembly to a vagus nerve of the patient;attaching a second stimulation electrode assembly to the vagus nerve of the patient; applying a first polarity to the first stimulation electrode assembly; applying a second polarity to the second stimulation electrode assembly; directionally stimulating the vagus nerve with with a first stimulation pattern transmitting by the electrode; and directionally stimulating the vagus nerve with a second stimulation pattern transmitted by the electrode.

A left and right nerve dual bipolar electrode treatment method for treating a plurality of conditions in a patient may comprise attaching a first stimulation electrode assembly to a first vagus nerve of the patient; attaching a second stimulation electrode assembly to a second vagus nerve of the patient; applying a first polarity to the first stimulation electrode assembly; applying a second polarity to the second stimulation electrode assembly; stimulating the vagus nerve with a first stimulation pattern transmitting by the first stimulation electrode assembly; and stimulating the vagus nerve with a second stimulation pattern transmitted by the second stimulation electrode assembly.

According to any one of the above-described treatment methods the first stimulation pattern may comprise a heart stimulation pattern; and wherein the second stimulation pattern comprises a brain stimulation pattern.

Any one of the above-described treatment methods may further comprise placing an EKG sensing electrode member in the patient so as to sense EKG signals; sensing EKG signals; and modifying one or both of the first and second stimulation patterns so as to adjust to the EKG signal.

### BRIEF DESCRIPTION OF THE DRAWINGS

The foregoing brief description as well as the following detailed description of the preferred embodiment of the invention will be better understood when read in conjunction with the appended drawings. It should be understood, however, that the invention is not limited to the precise arrangements and instrumentalities shown herein. The components in the drawings are not necessarily to scale, emphasis instead being placed upon clearly illustrating the principles of the present invention. Moreover, in the drawings, like reference numerals designate corresponding parts throughout the several views.

The invention may take physical form in certain parts and arrangement of parts. For a more complete understanding of the present invention, and the advantages thereof, reference is now made to the following descriptions taken in conjunction with the accompanying drawings, in which:

FIG. 1 illustrates a single bipolar electrode combination device implanted in a person;

FIG. 2 illustrates a single nerve dual bipolar electrode combination device implanted in a person;

FIG. 3 illustrates a right and left nerve dual bipolar combination device implanted in a person;

FIG. 4 is a schematic diagram of a single bipolar electrode combination device;

FIG. 5 is a schematic diagram of a dual bipolar electrode combination device;

FIG. 6 illustrates an interleaved signal flow;

FIG. 7 illustrates an asynchronous dual bipolar electrode flow;

FIG. 8 depicts a directional electrode assembly attached to a vagus nerve; and

FIG. 9 depicts an alternative directional electrode assembly attached to a vagus nerve.

### DETAILED DESCRIPTION OF EXEMPLARY EMBODIMENTS

According to some embodiments, the present invention provides a design for an implantable medical device that will switch stimulation polarity and will provide different stimulation parameters for CHF and stroke.

The patient will typically be treated for congestive heart failure with a first bi-polar electrode stimulating the right vagus nerve.

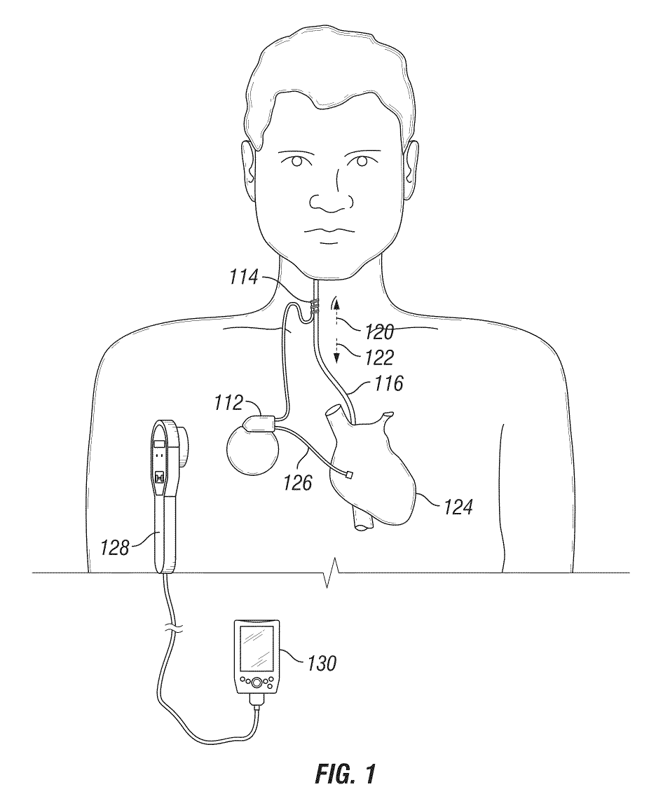

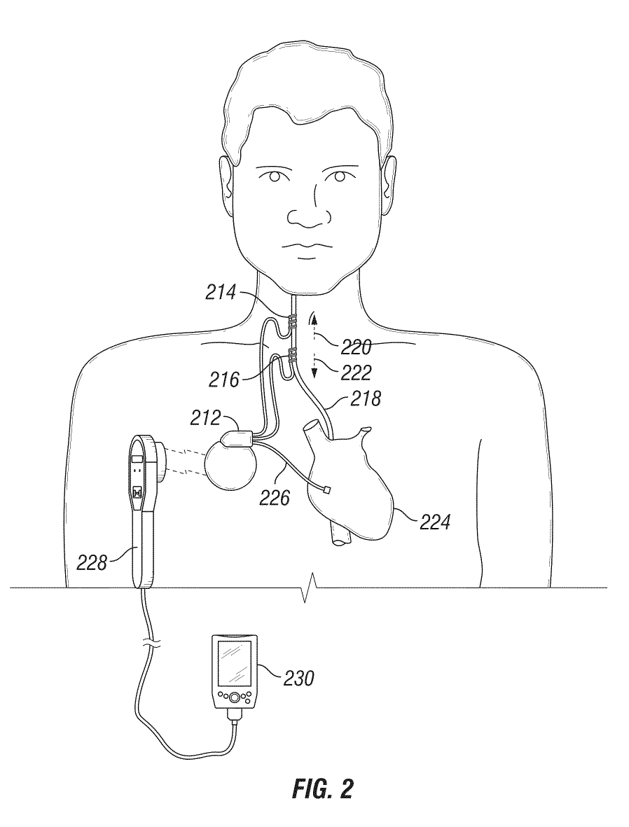

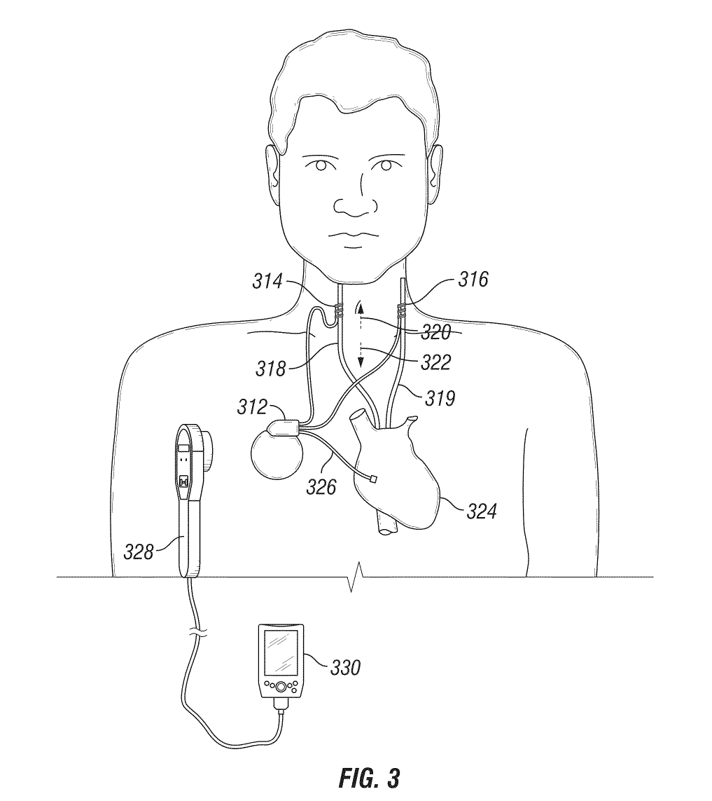

Stimulation with a specific stroke stimulation parameter could be applied by (a) reversing the polarity on the first right vagus nerve bi-polar electrode (FIGS. 1, 4, and 6), (b) providing a second bi-polar electrode and stimulation channel to stimulate the right vagus nerve (FIGS. 2, 5, and 6) or (c) providing a second bi-polar electrode and stimulation channel to stimulate the left vagus nerve (FIGS. 3, 5, and 7).

FIG. 1, illustrating a single bipolar electrode combination device implanted in a person, shows neurotransmitter 112, electrode assembly 114, vagus nerve 116, brain stimulating signal A 120, heart stimulating signal B 122, heart 124, EKG sensing electrode member 126, handheld programming wand 128, and handheld controller 130.

FIG. 2, illustrating a single nerve dual bipolar electrode combination device implanted in a person, shows neurotransmitter 212, first directional electrode assembly 214, second directional electrode 216, vagus nerve 218, brain stimulating signal 220, heart stimulating signal 222, heart 224, EKG sensing electrode member 226, handheld programming wand 228, and handheld controller 230.

FIG. 3, illustrating a right and left nerve dual bipolar combination device implanted in a person, shows neurotransmitter 312, first polarity electrode assembly 314, second polarity electrode assembly 316, right vagus nerve 318, right vagus nerve 319, brain stimulating signal 320, heart stimulating signal 322, heart 324, EKG sensing electrode member 326, handheld programming wand 328, and handheld controller 330.

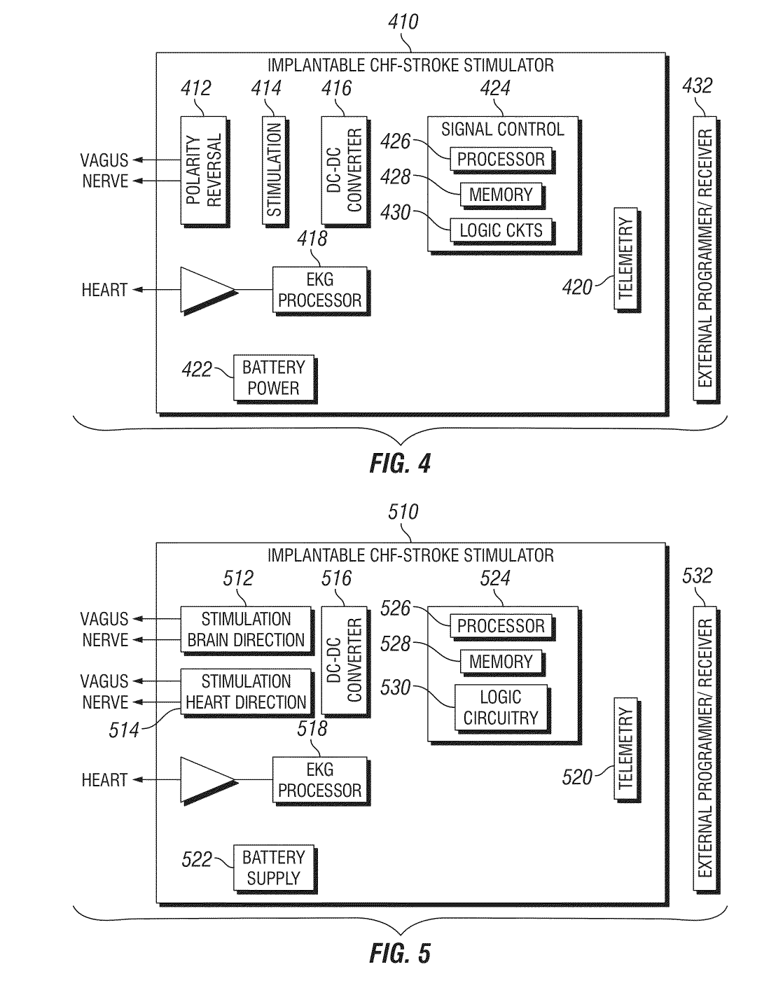

FIG. 4, a schematic diagram of a single bipolar electrode combination device, shows implantable congestive heart failure stimulator 410, polarity reversal unit 412, stimulation unit 414, DC-DC converter 416, EKG processor 418, telemetry unit 420, battery power 422, signal control 424, processor 426, memory 428, logic circuits 430, and external programmer/receiver 432. Electrodes extend (schematically shown by arrows) from polarity reversal unit 412.

FIG. 5, a schematic diagram of a dual bipolar electrode combination device, shows implantable congestive heart failure stimulator 510, brain directional stimulation unit 512, heart directional stimulation unit 514, DC-DC converter 516, EKG processor 518, telemetry unit 520, battery power 522, signal control 524, processor 526, memory 528, logic circuits 530, and external programmer/receiver 532. Electrodes extend (schematically shown by arrows) from brain directional stimulation unit 512. Further electrodes extend (schematically shown by arrows) from heart directional stimulation unit 514.

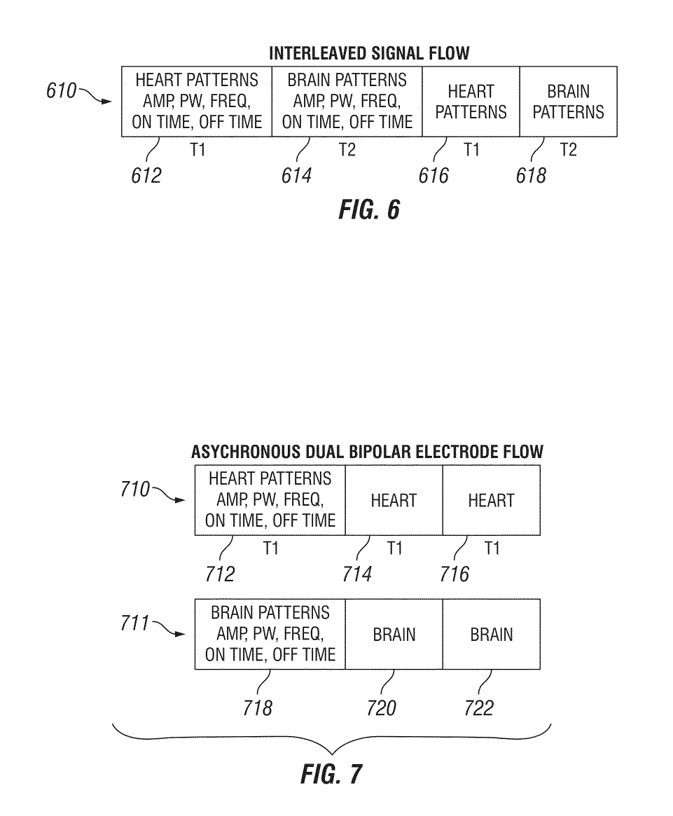

FIG. 6, illustrating an interleaved signal flow, shows pulse train 610, heart patterns pulse 612 for time T1, brain patterns pulse 614 for time T2, heart patterns pulse 616 for time T1, brain patterns pulse 618 for time T2.

FIG. 7, illustrating an asynchronous dual bipolar electrode flow, shows pulse train 710, pulse train 711, heart patterns pulse 712 for time T1, heart patterns pulse 714 for time T1, heart patterns pulse 716 for time T1, brain patterns pulse 718 for time T2, brain patterns pulse 720 for time T2, and brain patterns pulse 722 for time T2.

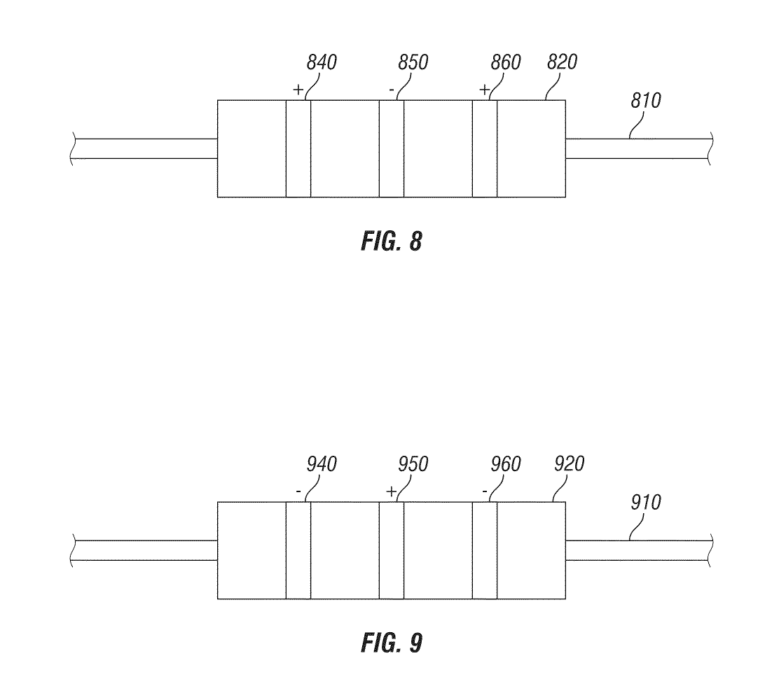

FIG. 8, depicting a directional electrode assembly attached to a vagus nerve, shows vagus nerve 810 and directional electrode 820. Tube 830 extends around vagus nerve 810. Inside the tube, conductors 840, 850, and 860 each encircle vague nerve 810 by 360°. Conductors 840 and 850 have opposing polarity. Conductors 850 and 860 have opposing polarity. FIG. 9, depicting an alternative directional electrode attached to a vagus nerve, shows vagus nerve 910 and alternate directional electrode assembly 920. Tube 930 extends around vagus nerve 910. Inside the tube, conductors 940, 950, and 960 each encircle vague nerve 910 by 360°. Conductors 940 and 950 have opposing polarity. Conductors 950 and 960 have opposing polarity. FIG. 9 differs from FIG. 8 in that the polarity of conductors 940, 950, and 960 is reversed from conductions 840, 850, and 860 respectively.

Referring to FIGS. 1 and 4, suitable methods and units for switching polarity are described in patent applications US 2010/0023090 and US 2011/0213437.

Referring to FIGS. 2 and 5, suitable methods and units for directional stimulation are described in patents U.S. Pat. No. 6,684,105 and U.S. Pat. No. 7,908,008.

Referring to FIGS. 6 and 7, it will be understood that amperage (amp), power (pw), frequency, (freq), on time, and off time are exemplary stimulation parameters. It will be understood that the stimulation parameters characterize a pulse pattern.

Referring to FIGS. 6 and 7, suitable methods and stimulation parameters for congestive heart failure are described in U.S. Pat. No. 6,622,041. In particular, techniques for increasing the inhibitory response of the parasympathetic or vagal system (decrease the autonomic nervous system drive) by appropriate stimulation of the cardiac branch of the vagus nerve are described in U.S. Pat. No. 6,622,041. In one method, the vagus nerve (left or right branch in the cervical region above the cardiac branch) is stimulated by the generator pulse waveform at a rate which is predetermined to limit the particular patient's upper heart rate to a physiologically safe limit within a prescribed range, e.g., 100-150 BPM. The stimulation rate is experimentally determined during treadmill testing of the patient. Typically, a vagus nerve stimulation rate of about 4 Hz limits the heart rate to about 100 BPM. Since the heart rate varies inversely with the VNS rate, lower stimulation rates produce proportionally higher heart rate limits. In an alternative method, heart rate is monitored and VNS is applied only when the heart rate is detected to fall below a threshold such as 100 BPM. Another alternative is to use feedback to automatically adjust the vagal stimulation rate to maintain the heart rate within the desired range.

Referring to FIGS. 6 and 7, suitable methods and stimulation parameters for stroke are described in US 20100069995. The stimulating electrical signal may supply a current to the cranial nerve in the range of from about 0.1 mA to about 10 mA for implantable electrodes and higher for transcutaneous stimulation. For example, the present inventor contemplates a typical current of about 1.25 mA. The stimulating electrical signal may comprise a train of pulses, each pulse having a pulse width ranging from about 50 microseconds to about 1,500 microseconds. For example, the present inventor contemplates a typical pulse width of about 250 microseconds. The stimulating electrical signal may comprise a train of pulses each pulse having a frequency ranging from about 1 Hz to about 200 Hz. For example, the present inventor contemplates a typical frequency of about 20 Hz. The stimulating electrical signal may be monophasic, biphasic, or a combination thereof. The electrical signal may comprise a train of pulses having a train duration ranging from about 5 milliseconds to about 8 hours, and each train may have different characteristics such as frequency and pulse width etc. For example, the electrical signal may comprise a plurality of trains of pulses, each train having a train duration ranging from about 5 milliseconds to about 8 hours. The plurality of trains may have an interval between trains ranging from about 0.25 seconds to about 1 month. The plurality of trains may be a plurality of intervals, each ranging from about 0.25 seconds to about 1 month. The trains may be supplied on demand.

When the patient has a stroke, stimulation with the specific stroke stimulation parameter could be initiated with a magnet, programming or some other type of communication. Suitable magnets and programming wands are described in U.S. Pat. No. 5,304,206. Detection of the stroke from the EKG and or 6 axis accelerometer would also be possible for some patients.

Referring to FIGS. 1, 4, and 6, when using the same electrode to stimulate the heart for CHF and the brain for stroke, the stimulation signals would desirably be interleaved, where the heart would be stimulated part of the time and the brain stimulated part of the time and the stimulation periods would not overlap. The electrode used for both CHF and stroke is desirably a standard nerve electrode and not one designed to preferentially stimulate in one direction. A suitable standard electrode is described in U.S. Pat. No. 4,573,481. The directionality preference of standard electrodes may be obtained by signal polarity. These could be switched during the cardiac cycle, during times when stimulation was stopped by design for congestive heart failure or it could be interleaved by longer periods of time.

It will be understood that an electrode assembly may comprise at least a plus and a minus electrode. A bipolar electrode assembly may comprise two electrodes. Suitable two electrode assemblies are described in U.S. Pat. No. 4,573,481. Referring to FIGS. 8 and 9, a directional electrode assembly may comprise three electrodes. Suitable three electrode assemblies are described in U.S. Pat. No. 4,573,481.

The times may be programmed to change during the time after the stroke. The stimulation parameters and/or the length of stimulation may be different during the acute stroke recovery period than the chronic recovery period. There may be several automatic adjustments during the recovery period. For example (a) acute recovery parameters during the first 24 hours, (b) sub-acute recovery parameters during the first several days after the stroke, (c) long term recovery parameters during the first 6 to 24 months after the stroke and (d) maintenance parameters following the long term recovery period (c). The parameters and time periods could be programmed to change automatically or the physician could manually reprogram for each period based on the patient's recovery progress.

Referring to FIGS. 1 and 4, a single bi-polar electrode with switched polarity has the advantage of simplicity for surgery and nerve exposure which should increase the reliability of the system and minimize surgical complications.

Referring to FIG. 2, a separate bi-polar electrode on either the right or left vagus nerve has the advantage of delivering stimulation independently of the congestive heat failure therapy. Referring to FIG. 6, it would still be desirable to interleave stimulation signals if both sets of bi-polar electrodes were implanted on the same nerve branch in order to avoid stimulation signal interference. Referring to FIG. 7, stimulation signals could be non-synchronous and overlap when the congestive heart failure electrodes are implanted on one cranial nerve and the stroke stimulation is implanted on the opposite cranial nerve.

Referring to FIG. 3, the time to implant on opposite nerves would likely be doubled in that configuration. Stimulation on opposite nerves may be preferred if there the length of nerve exposed to implant both sets of bi-polar electrodes on one side is greater than space available or longer than desired by the implanting physician.

Another design option would be to switch to uni-polar stimulation for congestive heart failure and or stroke therapy. In this example, the metal generator enclosure would be the return path, one electrode would be the congestive heart failure stimulation channel and one would be the stroke stimulation channel. However, stimulation directionality is not likely using uni-polar stimulation. The stimulation signals used to treat congestive heart failure may be too high to be tolerated for the stroke therapy. With this dual uni-polar electrode arrangement, the stimulation signals could be interleaved or asynchronous.

It should be understood that the stroke stimulation may be comprised of one or more sets of parameters or multiple electrodes in order to individually treat more than one symptom of stroke. This is described in the Danielsson application US 2010/0069995. The stroke sets of patterns could be delivered in series or could be separated with the congestive heart failure stimulation pattern between each individual stroke stimulation pattern.

Each of the references, patents, and patent applications cited herein is hereby incorporated herein by reference.

Although the invention has been described with reference to specific embodiments, these descriptions are not meant to be construed in a limiting sense. Various modifications of the disclosed embodiments, as well as alternative embodiments of the invention will become apparent to persons skilled in the art upon reference to the description of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

It is therefore, contemplated that the claims will cover any such modifications or embodiments that fall within the true scope of the invention.

1. An implantable medical device for treating a plurality of conditions of a patient, comprising:
a stimulation electrode assembly attachable to a vagus nerve, wherein the stimulation electrode assembly comprises first and second electrodes;
a polarity reversal unit in electrical communication with the stimulation electrode assembly; and
a stimulation unit in electrical communication with the polarity reversal unit, wherein the stimulation unit is capable to control interleaved signals of a first stimulation pattern transmittable by the first stimulation electrode and a second stimulation pattern transmittable by the second stimulation electrode.

2. The implantable medical device according to claim 1, further comprising:
a signal control processor in electrical communication with the stimulation unit.

3. The implantable medical device according to claim 1, wherein the plurality of conditions comprises congestive heart failure and stroke.

4. The implantable medical device according to claim 3, wherein the first stimulation pattern comprises a heart stimulation pattern; and wherein the second stimulation pattern comprises a brain stimulation pattern.

5. The implantable medical device according to claim 3, further comprising:
an EKG sensing electrode member; and
an EKG processor in electrical communication with the EKG sensing electrode member.

6. The implantable medical device according to claim 5, further comprising:
a signal control processor in electrical communication with the stimulation unit and the EKG processor.

7. A system for treating a plurality of conditions in a patient, comprising:
The the implantable medical device according to claim 8; and
a handheld programming wand, wherein the handheld programming wand is capable to change one or both of the first stimulation pattern and the second stimulation pattern.

8. An implantable medical device for treating a plurality of conditions in a patient, comprising:
a first stimulation electrode assembly attachable to a vagus nerve, wherein the first stimulation electrode assembly has a first polarity;
a second stimulation electrode assembly attachable to the vagus nerve, wherein the second stimulation electrode assembly has a second polarity;
a first stimulation unit in electrical communication with the first stimulation electrode assembly, wherein the first stimulation unit is capable to control a first directional stimulation pattern transmittable by the first stimulation electrode assembly; and
a second stimulation unit in electrical communication with the second directional stimulation electrode assembly, wherein the second stimulation unit is capable to control a second directional stimulation pattern transmittable by the second stimulation electrode assembly.

9. The implantable medical device according to claim 8, further comprising:
a signal control processor in electrical communication with each stimulation unit.

10. The implantable medical device according to claim 8, wherein the plurality of conditions comprises congestive heart failure and stroke.

11. The implantable medical device according to claim 10, wherein the first stimulation pattern comprises a heart stimulation pattern; and wherein the second stimulation pattern comprises a brain stimulation pattern.

12. The implantable medical device according to claim 10, further comprising:
an EKG sensing electrode member; and
an EKG processor in electrical communication with the EKG sensing electrode member.

13. The implantable medical device according to claim 12, further comprising:
a signal control processor in electrical communication with each stimulation unit and the EKG processor.

14. A system for treating a plurality of conditions in a patient, comprising:
the implantable medical device according to claim 8; and
a handheld programming wand, wherein the handheld programming wand is capable to change one or both of the first stimulation pattern and the second stimulation pattern.

15. An implantable medical device, comprising:
a first stimulation electrode assembly attachable to a first vagus nerve, wherein the first stimulation electrode assembly has a first polarity;
a second stimulation electrode assembly attachable to a second vagus nerve, wherein the second stimulation electrode assembly has a second polarity;
a first stimulation unit in electrical communication with the first stimulation electrode assembly, wherein the first stimulation unit is capable to control a first directional stimulation pattern transmittable by the first stimulation electrode assembly; and
a second stimulation unit in electrical communication with the second directional stimulation electrode assembly, wherein the second stimulation unit is capable to control a second directional stimulation pattern transmittable by the second stimulation electrode assembly.

16. The implantable medical device according to claim 15, further comprising:
a signal control processor in electrical communication with each stimulation unit.

17. The implantable medical device according to claim 15, wherein the plurality of conditions comprises congestive heart failure and stroke.

18. The implantable medical device according to claim 17, wherein the first stimulation pattern comprises a heart stimulation pattern; and wherein the second stimulation pattern comprises a brain stimulation pattern.

19. The implantable medical device according to claim 17, further comprising:
an EKG sensing electrode member; and
an EKG processor in electrical communication with the EKG sensing electrode member.

20. The implantable medical device according to claim 19, further comprising:
a signal control processor in electrical communication with each stimulation unit and the EKG processor.

21. A system for treating a plurality of conditions in a patient, comprising:
the implantable medical device according to claim 15; and
a handheld programming wand, wherein the handheld programming wand is capable to change one or both of the first stimulation pattern and the second stimulation pattern.

22. A treatment method for treating a plurality of conditions in a patient, comprising:
attaching a stimulation electrode assembly to a vagus nerve in the patient;
stimulating the vagus nerve with a first stimulation pattern transmitted by the electrode;
changing the polarity of the electrode assembly; and
stimulating the vagus nerve with a second stimulation pattern transmitted by the electrode assembly.

23. The treatment method according to claim 22, wherein the plurality of conditions comprises congestive heart failure and stroke.

24. The treatment method according to claim 23, wherein the first stimulation pattern comprises a heart stimulation pattern; and wherein the second stimulation pattern comprises a brain stimulation pattern.

25. The treatment method according to claim 23, further comprising:
placing an EKG sensing electrode member in the patient so as to sense EKG signals;
sensing EKG signals; and
modifying one or both of the first and second stimulation patterns so as to adjust to the EKG signal.

26. A treatment method for treating a plurality of conditions in a patient, comprising:
attaching a first stimulation electrode assembly to a vagus nerve of the patient;
attaching a second stimulation electrode assembly to the vagus nerve of the patient;
applying a first polarity to the first stimulation electrode assembly;
applying a second polarity to the second stimulation electrode assembly;
directionally stimulating the vagus nerve with with a first stimulation pattern transmitting by the electrode assembly; and
directionally stimulating the vagus nerve with a second stimulation pattern transmitted by the electrode assembly.

27. The treatment method according to claim 26, wherein the plurality of conditions comprises congestive heart failure and stroke.

28. The treatment method according to claim 27, wherein the first stimulation pattern comprises a heart stimulation pattern; and wherein the second stimulation pattern comprises a brain stimulation pattern.

29. The treatment method according to claim 28, further comprising:
placing an EKG sensing electrode member in the patient so as to sense EKG signals;
sensing EKG signals; and
modifying one or both of the first and second stimulation patterns so as to adjust to the EKG signal.

30. A treatment method for treating a plurality of conditions in a patient, comprising:
attaching a first stimulation electrode to a first vagus nerve of the patient;
attaching a second stimulation electrode assembly to a second vagus nerve of the patient;
applying a first polarity to the first stimulation electrode assembly;
applying a second polarity to the second stimulation electrode assembly;
stimulating the vagus nerve with a first stimulation pattern transmitting by the first stimulation electrode assembly; and
stimulating the vagus nerve with a second stimulation pattern transmitted by the second stimulation electrode assembly.

31. The treatment method according to claim 30, wherein the plurality of conditions comprises congestive heart failure and stroke.

32. The treatment method according to claim 31, wherein the first stimulation pattern comprises a heart stimulation pattern; and wherein the second stimulation pattern comprises a brain stimulation pattern.

33. The treatment method according to claim 31, further comprising:
placing an EKG sensing electrode member in the patient so as to sense EKG signals;
sensing EKG signals; and
modifying one or both of the first and second stimulation patterns so as to adjust to the EKG signal.
